(19) United States
(12) Patent Application Publication
(10) Pub. No.: US 2009/0274947 A1
(43) Pub. Date: Nov. 5, 2009

(54) Imidazolidinone nitroxides as electrode materials for energy storage devices

(76) Inventors: Peter Nesvadba, Marly (CH); Lucienne Bugnon, Pfeffingen (CH); Markus Frey, Rheinfelden (CH)

Correspondence Address: JoAnn Villamizar;Ciba Corporation/Patent Department, 540 White Plains Road, P.O. Box 2005, Tarrytown, NY, 10591, US

(21) Appl. No.: 12/310,701

(22) PCT Filed: Sep. 3, 2007
(86) PCT No.: PCT/EP2007/059156

§ 371 Date: Mar. 4, 2009

(30) Foreign Application Priority Data
Sep. 12, 2006 (EP) 06120493.9
Mar. 27, 2007 (EP) 07104943.1

(51) Int. Cl. H01M 10/00; C07D 471/10; C07D 498/10; C08F 26/06

(52) U.S. Cl. 429/50; 548/316.7; 546/20; 546/19; 526/260; 526/264

(57) ABSTRACT

The invention relates to a an electrical energy storage device, such as a capacitor or a secondary battery, utilizing as active element the oxidation and reduction cycle of a sterically hindered imidazolidinone nitroxide radical. Further aspects of the invention are a method for providing such an energy storage device, the use of the respective compounds as active elements in energy storage devices and selected novel imidazolidinone nitroxide compounds.

The invention relates to a an electrical energy storage device, such as an electrochemical capacitor or a secondary battery, utilizing as active element the oxidation and reduction cycle of a sterically hindered imidazolidinone nitroxide radical. Further aspects of the invention are a method for providing such an energy storage device, the use of the respective compounds as active elements in energy storage devices and selected novel imidazolidinone nitroxide compounds.

The use of various radicals, such as, for example, nitroxide radicals as active component in electrode materials of secondary batteries has been disclosed in EP 1 128 453. Since low solubility or insolubility of the electrode material in the battery electrolyte is preferred, polymeric or oligomeric nitroxides are of particular interest.

Nitroxide polymers as cathode active materials in organic radical batteries have already been disclosed, for example, in Electrochimica Acta 50, 827 (2004). The preparation of 4-meth-acryloyloxy-2,2,6,6-tetramethylpiperidine, its free radical polymerization and subsequent oxidation of the polymer into the corresponding polymeric nitroxide is described.

Due to the fast growing market of electronic devices, such as mobile telephones and mobile personal computers (lap-tops), there have been increasing needs in the last years for small and large-capacity secondary batteries with high energy density.

Today the most frequently used secondary battery for such applications is the lithium-ion secondary battery. Such a lithium-ion secondary battery uses a transition-metal oxide containing lithium in the positive electrode (cathode) and carbon in a negative electrode (anode) as active materials, and performs charge and discharge via insertion of Li in and elimination of Li from these active materials.

However, since the lithium-ion secondary battery uses a transition-metal oxide with a large specific gravity, particularly in the positive electrode, it has an undesirable secondary battery capacity per unit weight. There have been, therefore, attempts for developing a large-capacity secondary battery using a lighter electrode material. For example, U.S. Pat. Nos. 4,833 and 2,715,778 have disclosed a secondary battery using an organic compound having a disulfide bond in a positive electrode, which utilizes, as a principle of a secondary battery, an electrochemical oxidation-reduction reaction associated with formation and dissociation of a disulfide bond.

As mentioned above EP 1 128 453 similarly discloses, for example, nitroxide radicals as active components in electrode materials of secondary batteries.

Recently, a Chinese patent application CN 1741214-A disclosed that nitroxide radicals can also be used as an electrode material in supercapacitors.

Surprisingly it has now been found that imidazolidinone nitroxide radicals afford active electrode materials having an exceptionally high charge capacity. One aspect of the invention are, therefore, new imidazolidinone nitroxides and polymers derived therefrom having charge capacities up to around 200 Ah/kg and energy densities significantly superior as compared to the state of the art 2,2,6,6-tetramethyl piperidine N-oxide based polymers.

It has been surprisingly found that the voltage of a battery containing imidazolidinone nitroxides is higher as compared to the batteries described explicitly in the literature, which are based on 2,2,6,6-tetramethyl-piperidin-N-oxyl nitroxides (TEMPO). Moreover, the redox potential of imidazolidinone nitroxides can be tuned by virtue of their substitution pattern, thus allowing further increasing the battery voltage.

For example, the electromotoric force (EMF) of a TEMPO based battery is approximately 3.6 V. The EMF of an imidazolidinone nitroxide based battery can be significantly higher, compared with TEMPO based systems. This possible increase in energy content is clearly of high interest.

Additionally the imidazolidinone nitroxides show a fully reversible redox behavior when subjected to repeated oxidation into the corresponding oxoammonium salts and back-reduction into the nitroxide. This reversibility is a necessary condition for applicability in a secondary battery

Hence, the imidazolidinone nitroxides offer significant advantages when used as electrode materials in energy storage devices such as supercapacitors or secondary organic radical batteries.

One aspect of the invention is an electrical energy storage device with improved capacity, utilizing an electrode reaction of an active material in the reversible oxidation/reduction cycle in at least one of the positive or negative electrodes, which active material comprises a structural element of formula (I)

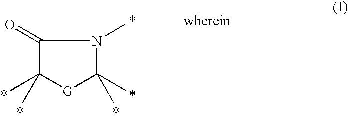

G is

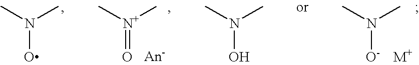

and * indicates a valence;
An⁻ is the anion of an organic or inorganic acid;

### M⁺ is Li⁺;

with the proviso that the structural element of formula (I) is not attached to a 1,3,5 triazine ring.

This invention provides an energy storage device, such as a secondary battery using a radical compound as an electrode active material. When the radical compound consists of lighter elements such as carbon, hydrogen and oxygen, it may be expected to provide a secondary battery with a high energy density per weight.

An electrode active material as used herein refers to a material directly contributing to an electrode reaction such as charge and discharge reactions, and plays a main role in a secondary battery system. An active material in this invention may be used as either a positive electrode or negative electrode active material, but it may be more preferably used as a positive electrode active material because it is characterized by a light weight and has a good energy density in comparison with a metal oxide system.

The underlying mechanism of energy storage is the reversible oxidation/reduction of the nitroxide radical according to Scheme 1:

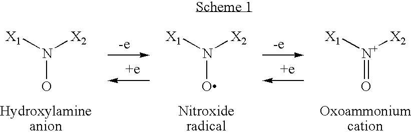

The counter ion of the oxoammonium cation, A⁻ may be, for example, the anion derived from LiPF₆, LiClO₄, LiBF₄, LiCF₃SO₃, LiN(CF₃SO₂)₂, LiN(C₂F₅SO₂)₂, LiC(CF₃SO₂)₃ and LiC(C₂FₛSO₂)₃.

Even though the use of the full redox window (hydroxylamine anion<—>oxoammonium cation) is possible, the currently preferred batteries use the redox pair nitroxide radical<—>oxoammonium cation. Hence, the electrons are exchanged between the oxidized state N⁺═O and reduced state N—O.

In this invention, a binder may be used for reinforcing binding between components.

Examples of a binder include polyvinylidene fluoride, a copolymer of vinylidene fluoride and hexafluoropropylene, a copolymer of vinylidene fluoride and tetrafluoroethylene, polytetrafluoroethylene, a copolymer rubber of styrene and butadiene, and resin binders such as polypropylene, polyethylene and polyimide.

According to the invention the active material in at least one of a positive electrode and a negative electrode comprises a radical compound without restrictions to its amount. However, since the capacity as a secondary battery depends on the amount of the radical compound contained in the electrode, the content is desirably 10 to 100% by weight, preferably 20 to 100% and in particular 50 to 100% for achieving adequate effects.

It is also possible to use more than one radical compound as active electrode material. The compound according to the invention may be mixed, for example, with a known active material to function as a complex active material.

When using the instant radical compound in a positive electrode, examples of materials for the negative electrode layer include carbon materials such as graphite and amorphous carbon, lithium metal or a lithium alloy, lithium-ion occluding carbon and conductive polymers. These materials may take an appropriate form such as film, bulk, granulated powder, fiber and flake.

A conductive auxiliary material or ion-conductive auxiliary material may also be added for reducing impedance during forming the electrode layer. Examples of such a material include carbonaceous particles such as graphite, carbon black and acetylene black and conductive polymers such as polyaniline, polypyrrole, polythiophene, polyacetylene and polyacene as conductive auxiliary materials as well as a gel electrolyte and a solid electrolyte as ion-conductive auxiliary material.

A catalyst may also be used for accelerating the electrode reaction. Examples of a catalyst include conductive polymers such as polyaniline, polypyrrole, polythiophene, polyacetylene and polyacene; basic compounds such as pyridine derivatives, pyrrolidone derivatives, benzimidazole derivatives, benzothiazole derivatives and acridine derivatives; and metal-ion complexes.

The concentration of the radical compound in this invention is preferably kept to 10¹⁹ spin/g or more, more preferably 10²¹ spin/g or more. With regard to the capacity of a secondary battery, as many spins/g as possible is desirable.

In general, a radical concentration may be expressed as a spin concentration. That is, a spin concentration means the number of unpaired electrons (radicals) per unit weight, which is determined by, for example, the following procedure from an absorption area intensity in an electron spin resonance spectrum (hereinafter, referred to as an “ESR” spectrum). First, a sample to be measured by ESR spectroscopy is pulverized by grinding it in, for example, a mortar, whereby the sample may be ground to a particle size in which skin effect, i.e., a phenomenon that microwave does not penetrate a sample, can be ignored. A given amount of the pulverized sample is filled in a quartz glass capillary with an inner diameter of 2 mm or less, preferably 1 to 0.5 mm, vacuumed to 10-5 mm Hg or less, sealed and subjected to ESR spectroscopy. ESR spectroscopy may be conducted in any commercially available model. A spin concentration may be determined by integrating twice an ESR signal obtained and comparing it to a calibration curve. There are no restrictions to a spectrometer or measuring conditions as long as a spin concentration can be accurately determined. For the stability of a secondary battery, a radical compound is desirably stable. A stable radical as used herein refers to a compound whose radical form has a long life time.

For example the structural element of formula (I) is of formulae (a1) or (a2)

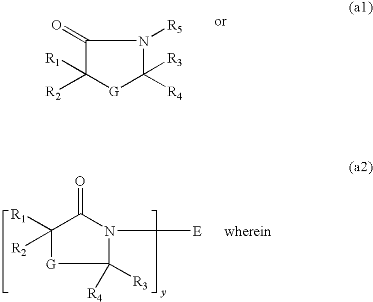

### G is

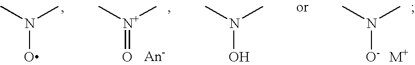

R₁, R₂, R₃ and R₄ are independently C₁-C₆alkyl, C₁-C₆alkyl, substituted by —COOM⁺, —COOR₆, —CONHR₆, —CON(R₆)₂, —OR₆, F, Cl, C₁-C₆alkyl interrupted by —O—, —NR₆—; or C₅-C₆cycloalkyl, C₃-C₆cycloalkylidene, C₇-C₉phenylalkyl, —COO⁻M⁺, —COOR₆, —CONHR₆, —CON(R₆)₂ or
R₁ and R₂ or R₃ and R₄, or R₁ and R₂ and R₃ and R₄ are a group

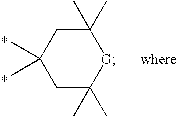

### M⁺ is Li⁺

An⁻ is the anion of an organic or inorganic acid;
R₆ is C₁-C₆alkyl, C₁-C₆alkyl substituted by —N₃; or C₂-C₆alkenyl, C₂-C₆alkinyl, glycidyl, C₅-C₆cycloalkyl, phenyl, C₇-C₉phenylalkyl or a group

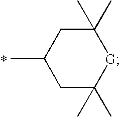

R₅ is H, C₁-C₆alkyl, C₂-C₆alkenyl, C₂-C₆alkinyl, glycidyl, C₅-C₆cycloalkyl, phenyl, C₇-C₉phenylalkyl, —O⁻M⁺, —OR₆, —OC(O)R₆, —C(O)R₆, —COOR₆, —CONHR₆, —CON(R₆)₂; or R₅ is C₁-C₆alkyl interrupted by —O—, —NR₆— or by a group

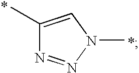

C₁-C₆alkyl, which is substituted by F, Cl, —COO⁻M⁺, —COOR₆, —CONHR₆, —CON(R₆)₂, OR₆, —OC(O)R₆, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
y is a number from 2 to 4;
when y is 2
E is a divalent group

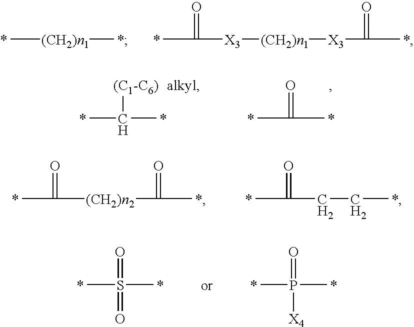

where n₁ is a number from 0 to 6 and n₂ is a number from 0 to 4; X₃ is —O—, —NH— or —NR₆—; X₄ is —OR₆, —NH₂, —NHR₆, or —N(R₆)₂;
when y is 3
E is a trivalent group

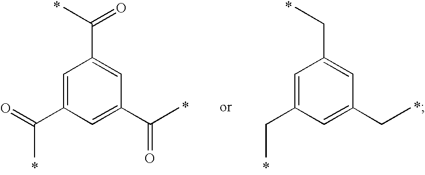

when y is 4
E is a tetravalent group of formula

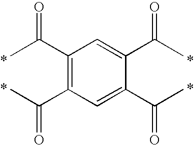

where * indicates a valence.

When R₅ is C₁-C₆alkyl which is interrupted by —O—, —NR₆— or by a group

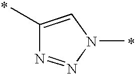

it can simultaneously also be substituted as defined above.

Suitable anions An⁻ are, for example derived from C₁-C₆carboxylic acids or from complex acids, such as LiPF₆, LiClO₄, LiBF₄, LiCF₃SO₃, LiN(CF₃SO₂)₂, LiN(C₂F₅SO₂)₂, LiC(CF₃SO₂)₃ and LiC(C₂FₛSO₂)₃.

For instance, G is

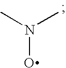

R₁, R₂, R₃ and R₄ are independently methyl, CF₃ or C₃-C₆cycloalkylidene; or
R₁ and R₂ or R₃ and R₄, or R₁ and R₂ and R₃ and R₄ are a group

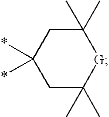

R₆ is C₁-C₆alkyl or C₂-C₆alkenyl;
R₅ is H, C₁-C₆alkyl, C₂-C₆alkenyl, C₂-C₆alkinyl, C₅-C₆cycloalkyl or —C(O)R₆; C₁-C₆alkyl, which is substituted by Cl;
y is 2;

E is a divalent group

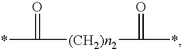

where n₂ is a number from 0 to 4;
where * indicates a valence.

For example, G is

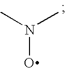

R₁, R₂, R₃ and R₄ are methyl; or
R₁ and R₂ are a group

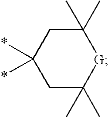

R₆ is C₁-C₆alkyl (e.g. methyl) or C₂-C₆alkenyl (e.g. C₃alkenyl);
R₅ is H, C₁-C₆alkyl (e.g. methyl), C₂-C₆alkenyl (e.g. vinyl), C₂-C₆alkinyl (e.g. propargyl) or —C(O)R₆; C₁-C₆alkyl (e.g. ethyl), which is substituted by Cl;
y is 2;

E is a divalent group

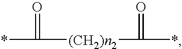

where n₂ is a number from 0 to 4 (e.g. 0);
where * indicates a valence.

For instance, the compound is of formula (a1)

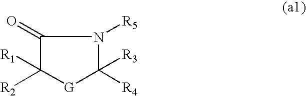

wherein

### G is

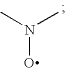

R₁, R₂, R₃ and R₄ are methyl, CF₃ or C₃-C₆cycloalkylidene; or
R₁ and R₂ or R₃ and R₄, or R₁ and R₂ and R₃ and R₄ are a group

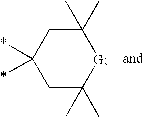

R₅ is H, methyl or C₅-C₆cycloalkyl.

In another embodiment of the invention the structural element of formula (I) is the repeating unit of a polymer and is of formulae (b1), (b2), (b3), (b4) or (b5)

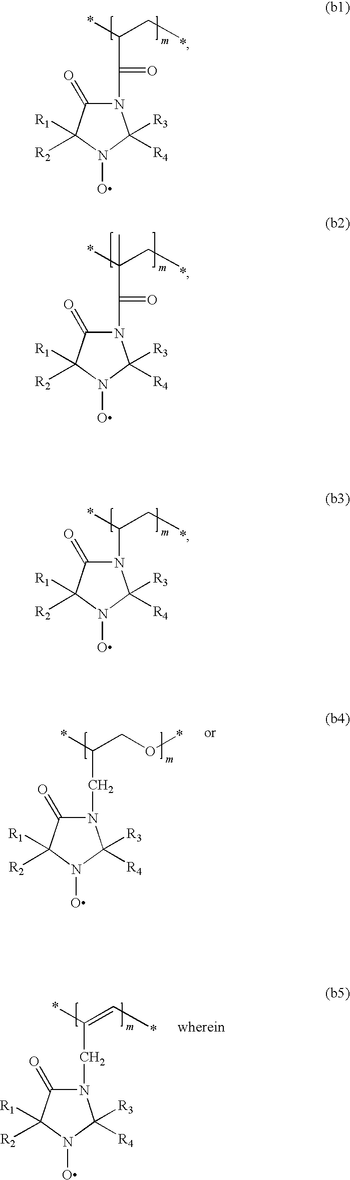

R₁, R₂, R₃ and R₄ are methyl, or C₃-C₆cycloalkylidene; or

R₁ and R₂ or R₃ and R₄, or R₁ and R₂ and R₃ and R₄ are a group

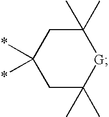

and the repeating index m is a number from 2 to 50 000, preferably 5 to 5000, most preferably 5 to 500.

For instance, the structural element of formula (I) is the repeating unit of a polymer and is of formula (b5)

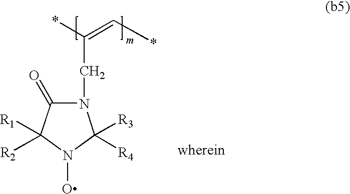

R₁, R₂, R₃ and R₄ are methyl; or
R₁ and R₂ are a group

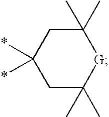

and the repeating index m is a number from 2 to 50 000, preferably 5 to 5000 most preferably 5 to 500.

The polymers or oligomers can be prepared by standard methods from the correspondingly functionalized monomers.

Preferably the electrical energy storage device is a secondary battery.

As outlined in Scheme 1 the underlying mechanism of energy storage is the reversible oxidation/reduction of the nitroxide radical. That means during charging and discharging always two species are present, namely the nitroxide radical and its oxidized or reduced form, depending on whether it is the active material of the positive or negative electrode.

Preferably the electrode reaction is that in the positive electrode.

In a preferred embodiment of a secondary battery G is a nitroxide radical

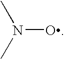

A preferred embodiment of the invention is an electrical energy storage device wherein the active material comprises from 10 to 100% by weight of the compound containing a structural element of formula (I).

Preferred is an electrical energy storage device wherein the active material has a spin concentration of at least 10²¹ spins/g.

A secondary battery according to this invention has a configuration, for example, as described in EP 1 128 453, where a negative electrode layer and a positive electrode layer are piled via a separator containing an electrolyte. The active material used in the negative electrode layer or the positive electrode layer is a radical compound with a structural element as described above.

In another configuration of a laminated secondary battery a positive electrode collector, a positive electrode layer, a separator containing an electrolyte, a negative electrode layer and a negative electrode collector are piled in sequence. The secondary battery may be a multi-layer laminate as well, a combination of collectors with layers on both sides and a rolled laminate.

The negative electrode collector and the positive electrode collector may be a metal foil or metal plate made of, for example, from nickel, aluminum, copper, gold, silver, an aluminum alloy and stainless steel; a mesh electrode; and a carbon electrode. The collector may be active as a catalyst or an active material may be chemically bound to a collector. A separator made of a porous film or a nonwoven fabric may be used for preventing the above positive electrode from being in contact with the negative electrode.

An electrolyte contained in the separator transfers charged carriers between the electrodes, i.e., the negative electrode and the positive electrode, and generally exhibits an electrolyte-ion conductivity of 10⁻⁵ to 10⁻¹ S/cm at room temperature. An electrolyte used in this invention may be an electrolyte solution prepared by, for example, dissolving an electrolyte salt in a solvent. Examples of such a solvent include organic solvents such as ethylene carbonate, propylene carbonate, dimethyl carbonate, diethyl carbonate, methyl ethyl carbonate, y-butyrolactone, tetrahydrofurane, dioxolane, sulforane, dimethylformamide, dimethylacetamide and N-methyl-2-pyrrolidone. In this invention, these solvents may be used alone or in combination of two or more. Examples of an electrolyte salt include LiPF₆, LiClO₄, LiBF₄, LiCF₃SO₃, LiN(CF₃SO₂)₂, LiN(C₂F₅SO₂)₂, LiC(CF₃SO₂)₃ and LiC(C₂F_{S}SO₂)₃.

An electrolyte may be solid. Examples of a polymer used in the solid electrolyte include vinylidene fluoride polymers such as polyvinylidene fluoride, a copolymer of vinylidene fluoride and hexafluoropropylene, a copolymer of vinylidene fluoride and ethylene, a copolymer of vinylidene fluoride and monofluoroethylene, a copolymer of vinylidene fluoride and trifluoroethylene, a copolymer of vinylidene fluoride and tetrafluoroethylene and a terpolymer of vinylidene fluoride, hexafluoropropylene and tetrafluoroethylene; acrylonitrile polymers such a copolymer of acrylonitrile and methyl methacrylate, a copolymer of acrylonitrile and methyl acrylate, a copolymer of acrylonitrile and ethyl methacrylate, a copolymer of acrylonitrile and ethyl acrylate, a copolymer of acrylonitrile and methacrylic acid, a copolymer of acrylonitrile and acrylic acid and a copolymer of acrylonitrile and vinyl acetate; polyethylene oxide; a copolymer of ethylene oxide and propylene oxide; and polymers of these acrylates or methacrylates. The polymer may contain an electrolyte solution to form a gel or the polymer may be used alone.

A secondary battery in this invention may have a conventional configuration, where, for example, an electrode laminate or rolled laminate is sealed in, for example, a metal case, a resin case or a laminate film made of a metal foil such as aluminum foil and a synthetic resin film. It may take a shape of, but not limited to, cylindrical, prismatic, coin or sheet.

A secondary battery according to this invention may be prepared by a conventional process. For example, from slurry of an active material in a solvent applied on an electrode laminate. The product is piled with a counter electrode via a separator. Alternatively, the laminate is rolled and placed in a case, which is then filled with an electrolyte solution. A secondary battery may be prepared using the radical compound itself or using a compound which can be converted into the radical compound by a redox reaction, as already described above.

The precursor compounds of the imidazolidinone nitroxides are essentially known and partially commercially available. All of them can be prepared by known processes. Their preparation is disclosed, for example, in: A. Khalaj et al., Monatshefte für Chemie, 1997, 128, 395-398; S. D. Worley et al., Biotechnol. Prog., 1991, 7, 60-66; T. Toda et al., Bull. Chem. Soc. Jap., 1972, 45, 557-561.

The oxidation may be carried out in analogy to the oxidation of 4-hydroxy-2,2,6,6-tetramethylpiperidine described in U.S. Pat. No. 5,654,434 with hydrogen peroxide. Another also suitable oxidation process is described in WO 00/40550 using peracetic acid.

An exhaustive description of the nitroxide chemistry can be found, for example, in L. B. Volodarsky, V. A. Reznikov, V. I. Ovcharenko.: “Synthetic Chemistry of Stable Nitroxides”, CRC Press, 1994.

The methods described in WO 2004/031150 can be used for the preparation of oxoammonium salts.

Further aspects of the invention are a method for providing an electrical energy storage device as described above, which method comprises incorporating an active material containing the structural element of formula (I) as defined above in at least one of the positive or negative electrodes;

and the use of a compound containing the structural element of formula (I) as an active material in at least one of the positive or negative electrodes of an electrical energy storage device.

Yet further aspects of the invention are novel nitroxyl radical compounds, which are particularly useful in the present invention.

For instance the compounds are of formulae (a1) or (a2)

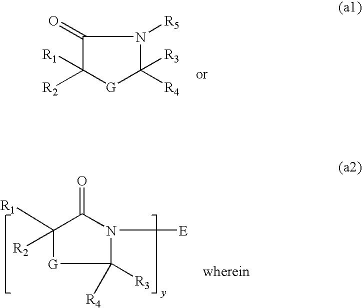

G is

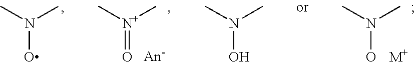

R₁, R₂, R₃ and R₄ are independently —COOH or —COO(C₁-C₆alkyl); or methyl, which can be substituted by F, Cl, OH, —COOH, —COO(C₁-C₆alkyl), —O—CO(C₁-C₆alkyl); or
R₁ and R₂ or R₃ and R₄, or R₁ and R₂ and R₃ and R₄ are a group

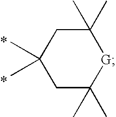

preferably R₁, R₂, R₃ and R₄ are methyl, which can be substituted by F, Cl, OH, —COOH, —COOCH₃, —O—COCH₃; or R₁ and R₂ or R₃ and R₄, or R₁ and R₂ and R₃ and R₄ are a group

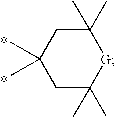

where
R₅ is H, C₁-C₆alkyl, C₂-C₆alkenyl, C₂-C₆alkinyl, glycidyl, C₅-C₆cycloalkyl, phenyl, C₇-C₉phenylalkyl, —O⁻M⁺, —OR₆, —OC(O)R₆, —OC(O)Cl, —C(O)Cl, —C(O)R₆, —COOR₆, —CONHR₆, —CON(R₆)₂; or
R₅ is C₁-C₆alkyl interrupted by —O—, —NR₆— or by a group

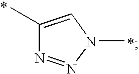

or C₁-C₆alkyl, which is substituted by F, Cl, —COO⁻M⁺, —COOR₆, —CONHR₆, —CON(R₆)₂, —C(O)C₁, OR₆, —OC(O)R₆, —OC(O)OR₆, —OC(O)Cl, —OC(O)NHR₆, —OC(O)N(R₆)₂, —NHC(O)R₆, —NR₆C(O)R₆, —NCO, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
for instance, R₅ is H, C₁-C₆alkyl, C₂-C₆alkenyl, C₂-C₆alkinyl, glycidyl, C₅-C₆cycloalkyl, phenyl, C₇-C₉phenylalkyl, —O⁻M⁺, —OR₆, —OC(O)R₆, —C(O)R₆, —COOR₆, —CONHR₆, —CON(R₆)₂; or R₅ is C₁-C₆alkyl interrupted by —O—, —NR₆— or by a group

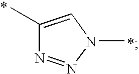

or C₁-C₆alkyl, which is substituted by F, Cl, —COO⁻M⁺, —COOR₆, —CONHR₆, —CON(R₆)₂, OR₆, —OC(O)R₆, —OC(O)OR₆, —OC(O)NHR₆, —OC(O)N(R₆)₂, —NHC(O)R₆, —NR₆C(O)R₆, —NCO, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
for example, R₅ is C₂-C₆alkenyl, C₂-C₆alkinyl, C₅-C₆cycloalkyl, C₇-C₉phenylalkyl, —O⁻M⁺, —OR₆, —OC(O)R₆, —OC(O)Cl, —C(O)Cl, —C(O)R₆, —COOR₆, —CONHR₆, —CON(R₆)₂; or R₅ is C₁-C₆alkyl interrupted by a group

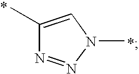

or C₁₋₆alkyl, which is substituted by —COO⁻M⁺, —COOR₆, —CONHR₆, —CON(R₆)₂, —C(O)C₁, OR₆, —OC(O)R₆, —OC(O)OR₆, —OC(O)Cl, —OC(O)NHR₆, —OC(O)N(R₆)₂, —NHC(O)R₆, —NR₆C(O)R₆, —NCO, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
for instance, R₅ is C₂-C₆alkenyl, C₂-C₆alkinyl, C₅-C₆cycloalkyl, C₇-C₉phenylalkyl, —O⁻M⁺, —OR₆, —OC(O)R₆, —OC(O)Cl, —C(O)Cl, —C(O)R₆, —COOR₆, —CONHR₆, —CON(R₆)₂; or R₅ is C₁-C₆alkyl interrupted by a group

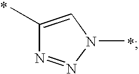

or C₁-C₆alkyl which is substituted by —COO⁻M⁺, —-CONHR₆, —CON(R₆)₂, —C(O)Cl, —OC(O)OR₆, —OC(O)Cl, —OC(O)NHR₆, —OC(O)N(R₆)₂, —NHC(O)R₆, —NR₆C(O)R₆, —NCO, NHC(O)NHR₆, —NR₆C(O)N(R₆)₂, —NHCOOR₆, —NR₆COOR₆, —N⁺(R₆)₃An⁻, S⁺(R₆)₂An⁻, P⁺(R₆)₃An⁻;
R₆ is —H, C₁-C₆alkyl, C₁-C₆alkyl substituted by —N₃; or C₂-C₆alkenyl, C₂-C₆alkinyl, glycidyl, C₅-C₆cycloalkyl, phenyl, C₇-C₉phenylalkyl or a group

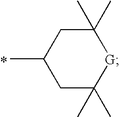

for instance R₆ is C₁-C₆alkyl substituted by —N₃; or C₂-C₆alkenyl, C₂-C₆alkinyl, C₅-C₆cycloalkyl, C₇-C₉phenylalkyl or a group

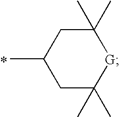

for example, R₆ is C₁-C₆alkyl or C₁-C₆alkyl substituted by —N₃, C₂-C₆alkenyl, C₂-C₆alkinyl, glycidyl, C₅-C₆cycloalkyl, C₇-C₉phenylalkyl or a group

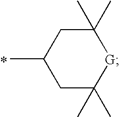

y is a number from 2 to 4;
when y is 2
E is a divalent group *—(CH₂)n₁—*;

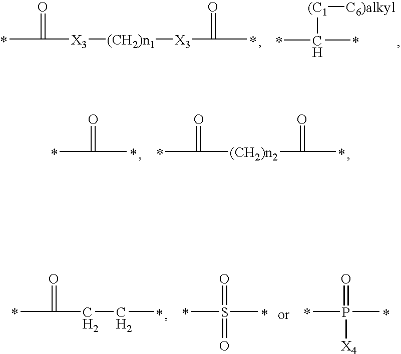

where n₁ is a number from 0 to 6 and n₂ is a number from 0 to 4; X₃ is —O—, —NH— or —NR₆—; X₄ is —OR₆, —NH₂, —NHR₆, or —N(R₆)₂;
when y is 3
E is a trivalent group

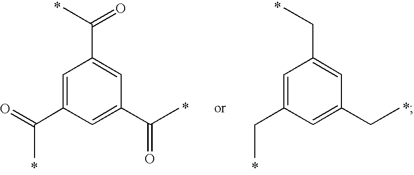

when y is 4
E is a tetravalent group of formula

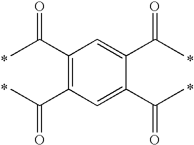

where * indicates a valence;
with the proviso that the following compounds are excluded

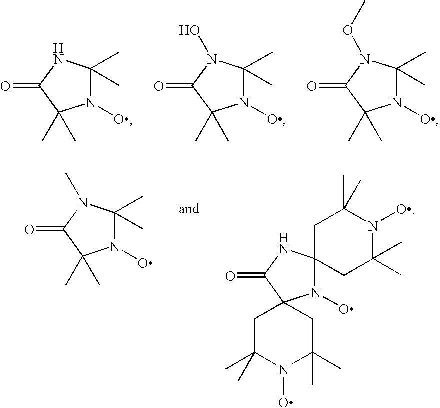

Preferred are compounds wherein

### G is

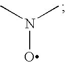

R₁, R₂, R₃ and R₄ are methyl, which can be substituted by F, Cl, OH, —COOH, —COOCH₃ or —O—COCH; or
R₁ and R₂ or R₃ and R₄, or R₁ and R₂ and R₃ and R₄ are a group

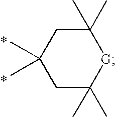

for example R₁, R₂, R₃ and R₄ are independently —COOH, —COO(C₁-C₆alkyl), methyl, which can be substituted by F, Cl, OH, —COOH, —COO(C₁-C₆alkyl), —O—CO(C₁-C₆alkyl) or R₁ and R₂or R₃ and R₄, or R₁ and R₂ and R₃ and R₄ are a group

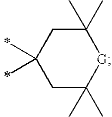

where
R₅ is H, C₁-C₆alkyl, C₂-C₆alkenyl, C₂-C₆alkinyl, glycidyl, C₅-C₆cycloalkyl, phenyl, C₇-C₉phenylalkyl, —OH, —OLi, OR₆, —C(O)R₆, —OC(O)R₆, —COOR₆, —CONHR₆, —CON(R₆)₂; or R₅ is C₁-C₆alkyl interrupted by —O— or —NR₆—; or C₁-C₆alkyl, which is substituted by F, Cl, —COO⁻M⁺, —COOR₆, —CONHR₆, —CON(R₆)₂, OH, OR₆, —OC(O)R₆, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
for example, R₅ is C₂-C₆alkenyl, C₂-C₆alkinyl, C₅-C₆cycloalkyl, C₇-C₉phenylalkyl, —OH, —OLi, OR₆, —C(O)R₆, —OC(O)R₆, —COOR₆, —CONHR₆, —CON(R₆)₂; or R₅ is C₁-C₆alkyl, which is substituted by —COO⁻M⁺, —COOR₆, —CONHR₆, —CON(R₆)₂, OH, OR₆, —OC(O)R₆, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;
for instance, R₅ is C₂-C₆alkenyl, C₂-C₆alkinyl, C₅-C₆cycloalkyl, C₇-C₉phenylalkyl, —OH, —OLi, OR₆, —C(O)R₆, —OC(O)R₆, —COOR₆, —CONHR₆, —CON(R₆)₂; or R₅ is C₁-C₆alkyl, which is substituted by —COO⁻M⁺, —CONHR₆, —CON(R₆)₂, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —NR₆COOR₆, —N⁺(R₆)₃An⁻, S⁺(R₆)₂An⁻, P⁺(R₆)₃An⁻;
for example, R₅ is H, C₁-C₆alkyl, C₂-C₆alkenyl, C₂-C₆alkinyl, glycidyl, C₅-C₆cycloalkyl, phenyl, C₇-C₉phenylalkyl, —OH, —OLi, OR₆, —C(O)R₆, —C(O)Cl, —OC(O)R₆, —COOR₆, —CONHR₆, —CON(R₆)₂; or R₅ is C₁-C₆alkyl interrupted by —O— or —NR₆—, C₁-C₆alkyl, which is substituted by F, Cl, —COO⁻M⁺, —COOR₆, —CONHR₆, —CON(R₆)₂, OH, OR₆, —OC(O)R₆, —OC(O)-halogen, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, P⁺(R₆)₃An⁻;

### M⁺ is Li⁺

An⁻ is the anion of an organic or inorganic acid;
R₆ is —H, C₁-C₆alkyl or C₁-C₆alkyl substituted by —N₃; or C₂-C₆alkenyl, C₂-C₆alkinyl, glycidyl, C₅-C₆cycloalkyl, C₇-C₉phenylalkyl or a group

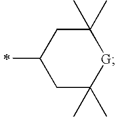

for example, R₆ is C₁-C₆alkyl substituted by —N₃; or C₂-C₆alkenyl, C₂-C₆alkinyl, C₅-C₆cycloalkyl, C₇-C₉phenylalkyl or a group

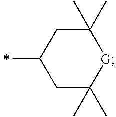

for instance, R₆ is C₁-C₆alkyl or C₁-C₆alkyl substituted by —N₃, C₂-C₆alkenyl, C₂-C₆alkinyl, glycidyl, C₅-C₆cycloalkyl, C₇-C₉phenylalkyl or a group

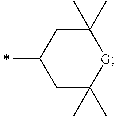

y is 2;
E is a divalent group

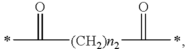

where n₂ is a number from 0 to 4;
with the proviso that the following compounds are excluded

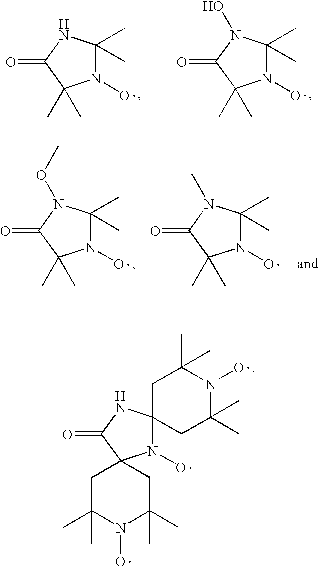

Most preferred are compounds wherein

### G is

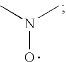

R₁, R₂, R₃ and R₄ are methyl; or
R₁ and R₂ are a group

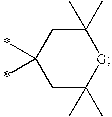

R₅ is H, C₂-C₆alkenyl (e.g. vinyl), C₂-C₆alkinyl (e.g. propargyl) or —C(O)R₆; or C₁-C₆alkyl (e.g. ethyl), which is substituted by Cl;
R₆ is C₁-C₆alkyl (e.g. methyl) or C₂-C₆alkenyl (e.g. C₃alkenyl);
y is 2;
E is a divalent group

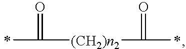

where n₂ is a number from 0 to 4 (e.g. 0);
with the proviso that the following compound is excluded

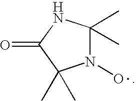

Particularly suitable are polymers with a repeating unit of formulae (b1), (b2), (b3), (b4) or (b5)

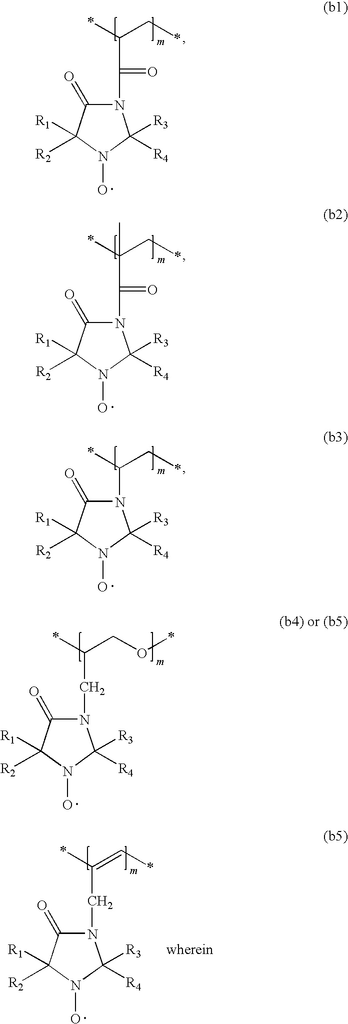

R₁, R₂, R₃ and R₄ are independently methyl, CF₃ or C₃-C₆cycloalkylidene; or
R₁ and R₂ or R₃ and R₄, or R₁ and R₂ and R₃ and R₄ are a group

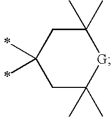

and the repeating index m is a number from 2 to 50 000 (e.g. 50 to 50 000), preferably 5 to 5000, most preferably 5 to 500.

Most particularly suitable are polymers with a repeating unit of formulae (b5)

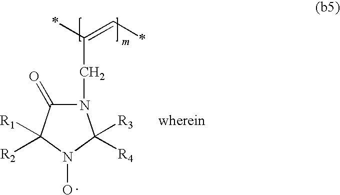

R₁, R₂, R₃ and R₄ are methyl; or
R₁ and R₂ are a group

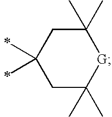

and the repeating index m is a number from 2 to 50 000 (e.g. 50 to 50 000), preferably 5 to 5000, most preferably 5 to 500.

Examples for individual compounds suitable in the instant invention are given in Table 1

TABLE 1

| | |
|---|---|
| 1 | 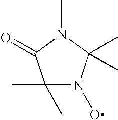 |
| 2 | 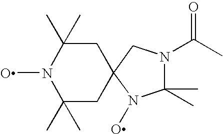 |
| 3 | 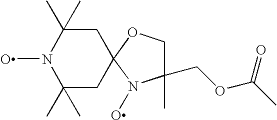 comparative |
| 4 | 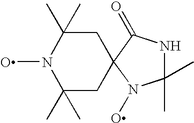 |
| 5 | 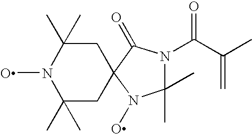 |
| 6 | 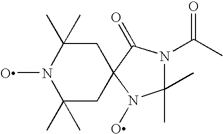 |
| 7 | 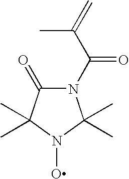 |
| 8 | 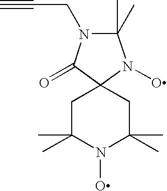 |
| 9 | 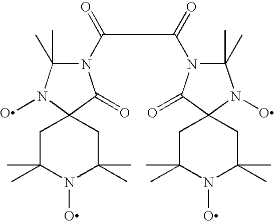 |
| 10 | 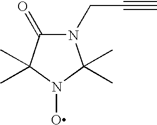 |
| 11 | 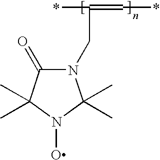 |
| 12 | 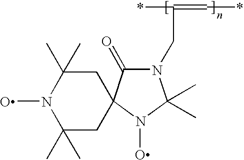 |
| 13 | 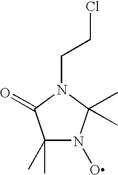 |
| 14 | 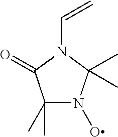 |

The definitions and preferences given above apply for all aspects of the invention.

The following examples illustrate the invention.

### A) PREPARATION EXAMPLES

### Example A1

### 2,2,3,5,5-Pentamethyl-imidazolidin-4-one-1-N-oxyl (Cmpd Nr. 1 of Table 1)

Hydrogen peroxide (aqueous, 30%, 2.5 g, 22 mmol) is slowly added to a solution of 2,2,3,5,5-pentamethyl-imidazolidin-4-one (1.85 g, 10 mmol) in acetic acid (15 ml) containing EDTA (0.0497 g, 0.17 mmol) and Na₂WO₄x2H₂O (0.0495 g, 0.15 mmol) and the resulting pale yellow suspension stirred overnight at room temperature (25° C.). Additional hydrogen peroxide (2.4 g, 21 mmol) is fed in and the orange solution stirred for another 2 days. The reaction mixture is brought to pH 7 (aqueous NaOH, 30%) and the resulting orange suspension extracted with CH₂Cl₂ (2×40 ml). The organic phase is brine-washed, dried over MgSO₄ and the solvent distilled off on a rotary evaporator to leave a reddish oil that solidified upon standing. Purification by chromatography (silica gel, hexane/ethylacetate 4/6) gives 0.4 g of the title compound as orange crystals, mp. 67-69° C. MS: for C₈H₁₅N₂O₂ (171.22) found M⁺=171.

### Intermediates:

### A) 2,2,5,5-Tetramethyl-imidazolidin-4-one

Prepared as described in EP-A-1283240 (2003; to D. Lazzari et al, Ciba Specialty Chemicals Holding Inc.; CAN 138:154404).

### B) 2,2,3,5,5-Pentamethyl-imidazolidin-4-one

Methyl iodide (3.6 g, 25 mmol) is slowly added to an ice-cooled suspension of 2,2,5,5-tetramethyl-imidazolidin-4-one (3.55 g, 25 mmol) in toluene (10 ml) containing potassium tert-butoxide (2.9 g, 25 mmol). The ice-bath is removed and the reaction mixture stirred overnight. Filtration and evaporation of the solvent on a rotary evaporator leaves a yellowish oil. Fractional short-path vacuum distillation using a Kugelrohr-oven affords 2 g of the title compound as a colourless liquid. MS: for C₈H₁₆N₂O (156.23) found M⁺=156. ¹H-NMR (300 MHz, CDCl₃), δ [ppm]: 2.81 (s, 3H), 1.78 (br s, 1H), 1.39 (s, 6H), 1.33 (s, 6H).

### Example A2

### 1-(2,2,7,7,9,9-Hexamethyl-1,3,8-triaza-spiro[4.5]dec-3-yl)-ethanone-1,8-N-oxyl (Cmpd Nr 2 of Table 1)

Hydrogen peroxide (aqueous, 30%, 0.61 g, 9 mmol) is slowly added to a solution of 1-(2,2,7,7,9,9-hexamethyl-1,3,8-triaza-spiro[4.5]dec-3-yl)-ethanone (0.53 g, 2 mmol) in water (2.5 ml) containing EDTA (0.01 g, 0.035 mmol) and Na₂WO₄x2H₂O (0.02 g, 0.06 mmol) and the resulting pale yellow suspension stirred overnight at room temperature (25° C.). Additional Na₂WO₄x2H₂O (0.02 g, 0.06 mmol) together with acetonitrile (1 g) are fed in and the orange solution stirred another 24 hours. The reaction mixture is extracted with CH₂Cl₂ (20 ml) and the organic phase washed with sodium hydroxide (aqueous, 1 molar) and brine. After drying over MgSO₄ the solvent is distilled off on a rotary evaporator to leave a red oil which solidifies upon addition of hexane. Fractional crystallization from hexane/ethylacetate gives 0.1 g the title compound as red solid, mp. 111-115° C. MS: for C₁₅H₂₇N₃O₃ (297.40) found M⁺=297.

### Intermediates:

### A) 4-Hydroxy-2,2,6,6-tetramethyl-piperidine-4-carbonitrile

Prepared as described in example A4 (intermediate A)

### B) 4-Amino-2,2,6,6-tetramethyl-piperidine-4-carbonitrile

Prepared as described in example A4 (intermediate B)

### C) N-(4-Cyano-2,2,6,6-tetramethyl-piperidin-4-yl)-acetamide

Acetic anhydride (99%, 3.88 g, 37.6 mmol) is slowly added to an ice-cooled solution of 4-amino-2,2,6,6-tetramethyl-piperidine-4-carbonitrile (92.5%, 7.37 g, 37.6 mmol) in CHCl₃ (40 ml). The ice-bath is removed and the reaction mixture stirred overnight. Ethanol (35 ml) is added to dissolve the solidified reaction mass and the solvent distilled off on a rotary evaporator. The remaining solid is dissolved in water (25 ml), brought to pH 12 (aqueous NaOH, 4 molar), saturated with NaCl and extracted with CH₂Cl₂ (50 ml). The organic phase is dried over Na₂SO₄ and the solvent distilled off on a rotary evaporator to leave 7.9 g of the title compound as an off-white solid, mp 153-160° C. MS: for C₁₂H₂₁N₃O (223.32) found M⁺=223. ¹H-NMR (300 MHz, CDCl₃), δ [ppm]: 5.90 (br s, 1H), 2.46 (d, J=13.5 Hz, 2H), 2.02 (s, 3H), 1.53 (d, J=13.5 Hz, 2H), 1.44 (s, 6H), 1.19 (s, 6H), 0.86 (br s, 1H).

### D) 4-Aminomethyl-2,2,6,6-tetramethyl-piperidin-4-ylamine

A mixture of N-(4-cyano-2,2,6,6-tetramethyl-piperidin-4-yl)-acetamide (6 g, 27 mmol), methanol (15 ml) and Raney-Ni (0.6 g) is hydrogenated 24 hours at 100° C./60 bar hydrogen pressure. After cooling down and releasing pressure the autoclave is discharged, the catalyst filtered off and the solvent evaporated to leave a yellow oil consisting of a mixture of 2,7,7,9,9-pentamethyl-1,3,8-triaza-spiro[4.5]dec-2-ene (major; MS: for C₁₂H₂₃N₃ (209.34) found M⁺=209) and N-(4-aminomethyl-2,2,6,6-tetramethyl-piperidin-4-yl)-acetamide (minor; MS: for C₁₂H₂₅N₃O (227.35) found M⁺=227). NaOH (aqueous, 30%, 36.5 g) is slowly added to the crude oil dissolved in methanol (26 g), the reaction mixture brought to reflux and stirred overnight. Methanol is distilled off, the remaining aqueous solution saturated with NaCl and extracted with diethylether. The organic phase is dried over Na₂SO₄ and the solvent distilled off on a rotary evaporator to leave an orange oil. Fractional short-path vacuum distillation using a Kugelrohr-oven affords 1.5 g of the title compound as a colourless, partially crystallizing liquid. MS: for C₁₀H₂₃N₃ (185.31) found M⁺=185. ¹³C-NMR (75 MHz, CDCl₃), δ [ppm]: 57.92, 52.95, 50.19, 46.12, 35.86, 31.59.

### E) 2,2,7,7,9,9-Hexamethyl-1,3,8-triaza-spiro[4.5]decane

Acetone (0.33 g, 5.7 mmol) is added to a suspension of 4-aminomethyl-2,2,6,6-tetramethyl-piperidin-4-ylamine (1.06 g, 5.7 mmol) in CHCl₃ (5.3 g) and the reaction mixture stirred at room temperature (25° C.) during 2.5 hours, during which the suspension turns into a solution. The solvent is evaporated on a rotary evaporator to leave 1.1 g of the title compound as a slightly yellowish oil. MS: for C₁₃H₂₇N₃ (225.38) found M⁺=225.

### F) 1-(2,2,7,7,9,9-Hexamethyl-1,3,8-triaza-spiro[4.5]dec-3-yl)-ethanone

Acetic anhydride (0.5 g, 5 mmol) is slowly added to an ice-cooled solution of 2,2,7,7,9,9-hexamethyl-1,3,8-triaza-spiro[4.5]decane (1.1 g, 5 mmol) in chloroform and the reaction mixture stirred during 2 hours. The solvent is distilled off on a rotary evaporator and the solid residue obtained taken up in water (8 ml). The solution is brought to pH 12 (aqueous NaOH, 4 molar), saturated with NaCl and extracted with CH₂Cl₂ (3×15 ml). The organic phase is dried over Na₂SO₄ and the solvent distilled off on a rotary evaporator to leave 1.2 g of the title compound as a slightly yellowish oil. MS: for C₁₅H₂₉N₃O (267.42) found M⁺=267.

### Example A3

### Acetic acid 3,7,7,9,9-pentamethyl-1-oxa-4,8-diaza-spiro[4.5]dec-3-yl-methyl ester-4,8-N-oxyl (Cmpd Nr. 3, comparative, of Table 1)

To a solution of acetic acid 3,7,7,9,9-pentamethyl-1-oxa-4,8-diaza-spiro[4.5]dec-3-yl-methyl ester (9.6 g, 0.033 mol, prepared as described under B)) in 50 ml dichloromethane is added together with solid NaHCO₃ (17 g, 0.2 mol) and 25 ml water. Peracetic acid (21.8 g, 40% in acetic acid, 0.115 mol) is then added during 20 minutes dropwise to the stirred mixture which is thereafter left stirring for 17 h at room temperature. The solution 2 M Na₂CO₃ is then added, the organic layer is separated, washed 3× with 10 ml water, dried over MgSO₄ and evaporated. The residue is purified by chromatography on silica gel (hexane-EtOAc 2:1) and crystallized from dichloromethane-hexane to afford 5.72 g of the title compound as red crystals, mp. 93-95° C. For C₁₅H₂₆N₂O₅ (314.38) calc./found C, 57.31/57.39; H, 8.34/8.53, N, 8.91/8.88. MS: M⁺=314.

### Intermediates

### A) (3,7,7,9,9-Pentamethyl-1-oxa-4,8-diaza-spiro[4.5]dec-3-yl)-methanol

Triacetonamine (62.1 g. 0.4 mol) and 2-amino-2-methyl-1,3-propandiol (21.0 g, 0.2 mol) are refluxed in 100 ml xylene on a Dean-Stark water separator during 11 h. The reaction mixture is cooled to room temperature, the precipitated solid is filtered off, rinsed with 50 ml toluene and discarded. The filtrate is evaporated on a rotary evaporator and the residue (67.7 g) is distilled under reduced pressure (0.01 mbar, 30-40° C.) to afford 37.4 g of a yellow, viscous liquid. Crystallisation from hexane at −20° C. affords 15.3 g of the title compound as colorless crystals, mp. 90-92° C. MS: for C₁₃H₂₆N₂O₂ (242.36) found M⁺=242.

### B) Acetic acid 3,7,7,9,9-pentamethyl-1-oxa-4,8-diaza-spiro[4.5]dec-3-yl-methyl ester

To a solution of 4-dimethylaminopyridine (0.37 g) and 3,7,7,9,9-pentamethyl-1-oxa-4,8-diaza-spiro[4.5]dec-3-yl)-methanol (9 g, 0.037 mol) in 30 ml dichloromethane is added at 0° C. the solution of acetyl chloride (3.2 g, 0.041 mol) in 5 ml dichloromethane. The mixture is stirred 1 h at room temperature, thereafter the solution of 1.8 g NaOH in 20 ml water is added. The organic layer is separated, washed 2× with 10 ml water, dried over MgSO₄ and evaporated to afford 9.75 g of the title compound as a yellow oil.

### Example A4

### 2,2,7,7,9,9-Hexamethyl-1,3,8-triaza-spiro[4.5]decan-4-one-1,8-N-oxyl (Cmpd Nr 4 of Table 1)

A 750 ml flask is charged with 2,2,7,7,9,9-hexamethyl-1,3,8-triaza-spiro[4.5]decan-4-one (23.95 g, 0.1 mol, prepared as described under D)), 250 ml dichloromethane, 60 ml water and NaHCO₃ (50.4 g, 0.6 mol). Peracetic acid (60.8 g, 40% in acetic acid, 0.32 mol) is then added during 25 minutes dropwise and at 5° C. to the stirred mixture which is thereafter left stirring for 3 h at room temperature. Additional peracetic acid (22.9 g, 0.12 mol) is then added and the stirring is continued for 15 h at room temperature. Further peracetic acid (5.73 g, 0.03 mol) is added and stirring is continued for 24 h. The organic layer is then separated, washed with 50 ml 2M-Na₂CO₃, dried over MgSO₄ and evaporated. The residue is crystallized from methanol to afford 20.7 g of the title compound as red crystals, m.p. 132-134° C. MS: for C₁₃H₂₃N₃O₃ (269.35) found M⁺=269.

### Intermediates

### A) 4-Hydroxy-2,2,6,6-tetramethyl-piperidine-4-carbonitrile

A 750 ml four neck flask is charged with triacetonamine (155.2 g, 1 mol) and acetone cyanohydrin (154.4 g, 1.81 mol). The suspension is stirred at 75-80° C. during 1 h and the acetone generated in the reaction is continuously distilled off. The mixture is then cooled to room temperature and 100 ml of methyl-t-butyl ether are added. The slurry is cooled to 5° C., filtered, washed with 150 ml cold methyl-t-butyl ether and dried to afford 149.7 g of the title compound as white crystals.

### B) 4-Amino-2,2,6,6-tetramethyl-piperidine-4-carbonitrile

To a methanolic solution of NH₃ gas (230 g, 16.6% NH₃ by weight) 4-hydroxy-2,2,6,6-tetramethyl-piperidine-4-carbonitrile (148.8 g, 0.816 mol) is added and the suspension is stirred at room temperature during 17 h. The colorless solution formed is evaporated at reduced pressure and at max. 25° C. to afford 163 g of the crude title compound as a colorless oil slowly crystallizing on standing.

### C) 4-Amino-2,2,6,6-tetramethyl-piperidine-4-carboxylic acid amide

A 1500 ml flask is charged with water (16.8 ml) and H₂SO₄ (420 ml, 98%, 7.73 mol). The acid is cooled to 10° C. and 4-amino-2,2,6,6-tetramethyl-piperidine-4-carbonitrile (160 g, ˜0.8 mol, crude) is added slowly with intense stirring during 1 h while keeping the temperature below 40° C. The mixture is then stirred for 23 h at 50° C., cooled to 35° C. and poured on 3.5 kg of ice. Solid NaOH (640 g, 16 mol) is added and the solution is extracted succesively with: 300 ml THF+200 ml EtOAc, 250 ml THF+100 ml EtOAc, 350 ml THF, 250 ml THF+100 ml EtOAc, 300 ml EtOAc. The combined extracts were washed with 100 ml of saturated NaCl, dried over K₂CO₃ and evaporated. The residue is triturated with cold EtOAc to afford 87.1 g of the title compound as colorless crystals, m.p. 142-144° C. MS: for C₁₀H₂₁N₃O (199.3) found M⁺=199.

### D) 2,2,7,7,9,9-Hexamethyl-1,3,8-triaza-spiro[4.5]decan-4-one

A 250 ml autoclave is charged with 4-amino-2,2,6,6-tetramethyl-piperidine-4-carboxylic acid amide (40 g, 0.2 mol), acetone (46.4 g, 0.8 mol), acetone dimethylketal (25 g, 0.24 mol) and Fulcat 22B catalyst (4 g). The mixture is then heated 17 h at 150° C., then cooled, dissolved in 800 ml methanol and filtered. The filtrate is diluted with 300 ml EtOAc and then evaporated to 188 g. The crystal slurry is cooled to 3° C., filtered, washed with 50 ml cold EtOAc and dried to afford 43.6 g of the title compound as white crystals, m.p. 247-250° C. MS: for C₁₃H₂₅N₃O (239.36) found M⁺=239.

### Example A5

### 2,2,7,7,9,9-Hexamethyl-3-(2-methyl-acryloyl)-1,3,8-triaza-spiro[4.5]decan-4-one-1,8-N-oxyl (Cmpd Nr. 5 of Table 1)

To a solution of 2,2,7,7,9,9-hexamethyl-1,3,8-triaza-spiro[4.5]decan-4-one-1,8-N-oxyl (0.94 g, 0.0035 mol), 0.021 g 4-dimethylaminopyridine and triethylamine (0.56 ml, 0.004 mol) in 12 ml dichloromethane is dropwise added methacryloylchloride (0.4 g, 0.0038 mol) at 2° C. The mixture is stirred 3 h at room temperature, then washed 3× with 5 ml water, the organic layer is dried over MgSO₄ and evaporated. Crystallisation from methanol affords 0.95 g of the title compound as red crystals, m.p. 108-110° C.

### Example A6

### 3-Acetyl-2,2,7,7,9,9-hexamethyl-1,3,8-triaza-spiro[4.5]decan-4-one-1,8-N-oxyl (Cmpd Nr. 6 of Table 1)

To a solution of 2,2,7,7,9,9-hexamethyl-1,3,8-triaza-spiro[4.5]decan-4-one-1,8-N-oxyl (2.7 g, 0.01 mol), 0.069 g 4-dimethylaminopyridine and triethylamine (1.5 ml, 0.0108 mol) in 30 ml dichloromethane is dropwise added acetyl chloride (0.86 g, 0.011 mol) at 3° C. The mixture is stirred 10 h at room temperature, then washed 3× with 15 ml water, the organic layer is dried over MgSO₄ and evaporated. Chromatography on silica gel (CH₂Cl₂-EtOAc (8:1) and crystallisation from methanol affords 2.12 g of the title compound as red crystals, m.p. 124-127° C.

### Example A7

### 2,2,5,5-Tetramethyl-3-(2-methyl-acryloyl)-imidazolidin-4-one-1-N-oxyl (Cmpd Nr. 7)

Methacryloylchloride (1.27 g, 12.1 mmol) is slowly added at 0-5° C. to a solution of 2,2,5,5-tetramethyl-imidazolidin-4-one-1-N-oxyl (1.73 g, 11 mmol), triethylamine (1.7 ml, 12.1 mmol) and 4-dimethylaminopyridine (67 mg) in dichloromethane (12 ml). The mixture is then stirred at room temperature for 2 h, washed with water (3×10 ml), dried over MgSO₄ and then the solvent is evaporated. The residue is recrystallized from methanol to afford 2.0 g of the title compound as red crystals, m.p. 93-96° C. MS for C₁₁H₁₇N₂O₃ [225.2] found MH⁺=226.

### Intermediates

A) 2,2,5,5-Tetramethyl-imidazolidin-4-one-1-N-oxyl, prepared as described by: Todda et al.: Bull. Chem. Soc. Jap. 45,1802 (1972)

### Example A8

### 2,2,7,7,9,9-Hexamethyl-3-prop-2-ynyl-1,3,8-triaza-spiro[4.5]decan-4-one-1,8-N-oxyl (Cmpd Nr. 8)

Sodium hydride (0.46 g, 10.5 mmol, 55% in mineral oil) is added to the solution of 2,2,7,7,9,9-hexamethyl-3,8-triaza-spiro[4.5]decan-4-one-1,8-N-oxyl (cmpd 4, 2.7 g, 10 mmol) in dry dimethyl formamide (60 ml) and the mixture is stirred 1 h at 40° C. It is then cooled to 3° C. and propargyl bromide (1.35 g, 11 mmol) is added slowly. The mixture is stirred 2 h at room temperature and then diluted with water (250 ml). The precipitate is filtered off, dried and recrystallized from dichloromethane-hexane to afford 2.8 g of the title compound as red crystals, m.p. 139-141° C. MS for C₁₆H₂₅N₃O₃ [307.4] found MH⁺=308. ATR-IR: —C≡C—H at 3253 cm⁻¹, >C═O at 1705 cm⁻¹.

### Example A9

### 1,2-Bis-(2,2,7,7,9,9-hexamethyl-4-oxo-1,3,8-triaza-spiro[4.5]dec-3-yl-1,8-N-oxyl)-ethane-1,2-dione (Cmpd Nr. 9)

Oxalylchloride (0.51 g, 4 mmol) is slowly added at 5° C. to the solution of 2,2,7,7,9,9-hexamethyl-3,8-triaza-spiro[4.5]decan-4-one-1,8-N-oxyl (cmpd 4, 2.4 g, 9 mmol), triethylamine (1.4 ml, 10 mmol) and 4-dimethylaminopyridine (200 mg) in dichloromethane (25 ml). The mixture is stirred for 22 h at room temperature, then washed with water (3×20 ml), dried over MgSO₄ and then the solvent is evaporated. The residue is recrystallized from methanol to afford 1.88 g of the title compound as red crystals, m.p. 195-197° C. MS for C₂₈H₄₄N₆O₈ [592.4] found MH⁺=593.

### Example A10

### 2,2,5,5-Tetramethyl-3-prop-2-ynyl-imidazolidin-4-one-1-N-oxyl (Cmpd Nr. 10)

Sodium hydride (0.7 g, 15.75 mmol, 55% in mineral oil) is added to the solution of 2,2,5,5-tetramethyl-imidazolidin-4-one-1-N-oxyl (example 7, intermediate A, 2.36 g, 15 mmol) in dry dimethyl formamide (15 ml) and the mixture is stirred 1.5 h at 40° C. It is then cooled to 3° C. and propargyl bromide (1.96 g, 16.5 mmol) is added slowly. The mixture is stirred 2 h at room temperature and then diluted with water (150 ml). The precipitate is filtered off, dried and recrystallized from dichloromethane-hexane to afford 1.5 g of the title compound as red crystals, m.p. 119-121° C. MS for C₁₀H₁₅N₂O₂ [195.2] found MH⁺=196. ATR-IR: —C≡C—H at 3233 cm⁻¹, >C═O at 1696 cm⁻¹.

### Example A11

### Poly(2,2,5,5-tetramethyl-3-prop-2-ynyl-imidazolidin-4-one-1-N-oxyl) (Cmpd Nr. 11)

### A) Non-Crosslinked Polymer

The solution of 2,2,5,5-tetramethyl-3-prop-2-ynyl-imidazolidin-4-one-1-N-oxyl (cmpd 10, 1.952 g, 10 mmol) in dimethylformamide (20 ml) is purged with argon for 10 minutes. The catalyst, Rh(norbornadiene)B(C₆H₅)₄ (52 mg. 0.1 mmol, prepared as described by: R. R. Schrock, J. A. Osborn, Inorg. Chem. 9, 2339, (1970)) is then added and the mixture is stirred for 17 h under argon at room temperature. It is then poured into methanol (200 ml), stirred for 2 h, the orange precipitate is filtered off, washed with methanol and dried 72 h at 50° C./100 mbar to afford 1.94 g of the title polymer as an orange powder.

### B) Crosslinked Polymer

The solution of 2,2,5,5-tetramethyl-3-prop-2-ynyl-imidazolidin-4-one-1-N-oxyl (cmpd 10, 2.93 g, 15 mmol) and the crosslinker N,N′-propargyloxalamide (74 mg, 0.45 mmol, prepared as described by: H. Reimlinger.: Justus Liebigs. Ann. Chem. 713, 113 (1968)) in dimethylformamide (25 ml) is purged with argon for 10 minutes. The catalyst, Rh(norbornadiene)B(C₆H₅)₄ (77 mg. 0.15 mmol, prepared as described by: R. R. Schrock, J. A. Osborn, Inorg. Chem. 9, 2339, (1970)) is then added and the mixture is stirred for 20 h under argon at room temperature. The red gel is then transferred into dichloromethane (100 ml) and stirred for 4 h. The precipitate is filtered off, redispersed in methanol (100 ml) and stirred for 4 h. The orange precipitate is filtered off, again redispersed in methanol, stirred for 12 h, filtered off, washed with methanol and dried 72 h at 50° C./100 mbar to afford 2.94 g of the title polymer as an orange powder.

ATR-IR: non-crosslinked polymer: —C≡C—H absorption absent, >C═O at 1705 cm⁻¹; crosslinked polymer: —C≡C—H absorption absent, >C═O at 1700 cm⁻¹. The absence of the —C≡C—H absorption band at 3233 cm⁻¹, which is observed in the monomer (cmpd 10), indicates a successful polymerization.

TGA (25-350° C. at 10° C./min under nitrogen): non-crosslinked polymer: practically no mass loss up to 190° C., decomposition between 200-350° C.; crosslinked polymer: practically no mass loss up to 190° C., decomposition between 200-350° C.

### Example A12

### Poly(2,2,7,7,9,9-hexamethyl-3-prop-2-ynyl-1,3,8-triaza-spiro[4.5]decan-4-one-1,8-N-oxyl), (Cmpd Nr. 12)

### A) Non-Crosslinked Polymer

The solution of 2,2,7,7,9,9-hexamethyl-3-prop-2-ynyl-1,3,8-triaza-spiro[4.5]decan-4-one-1,8-N-oxyl (cmpd 8, 1.537 g, 5 mmol) in dimethylformamide (10 ml) is purged with argon for 10 minutes. The catalyst, Rh(norbornadiene)B(C₆H₅)₄ (52 mg. 0.1 mmol, prepared as described by: R. R. Schrock, J. A. Osborn, Inorg. Chem. 9, 2339, (1970)) is then added and the mixture is stirred for 20 h under argon at 40° C. The mixture is then poured into methanol (250 ml), stirred for 2 h, the orange precipitate is filtered off, washed with methanol and dried 12 h at 50° C./100 mbar to afford 1.49 g of the title polymer as an orange powder.

### B) Crosslinked Polymer

The solution of 2,2,7,7,9,9-hexamethyl-3-prop-2-ynyl-1,3,8-triaza-spiro[4.5]decan-4-one-1,8-N-oxyl (cmpd 8, 3.074 g, 10 mmol) and the crosslinker N,N′-propargyloxalamide (49.2 mg, 0.3 mmol, prepared as described by: H. Reimlinger.: Justus Liebigs. Ann. Chem. 713, 113 (1968)) in dimethylformamide (25 ml) is purged with argon for 15 minutes. The catalyst, Rh(norbornadiene)B(C₆H₅)₄ (51 mg. 0.1 mmol, prepared as described by: R. R. Schrock, J. A. Osborn, Inorg. Chem. 9, 2339, (1970)) is then added and the mixture is stirred for 23 h under argon at room temperature. The red gel is then transferred into water (400 ml) and stirred for 3 h. The precipitate is filtered off, redispersed in methanol (300 ml) and stirred for 90 h. The orange precipitate is filtered off, washed with methanol and dried 72 h at 50° C./100 mbar to afford 2.9 g of the title polymer as an orange powder.

ATR-IR: non-crosslinked polymer: —C≡C—H absorption absent, >C═O at 1705 cm⁻¹; crosslinked polymer: —C≡C—H absorption absent, >C═O at 1705 cm⁻¹. The absence of the —C≡C—H absorption band at 3253 cm⁻¹, which is observed in the monomer (cmpd 8), indicates a successful polymerization.

TGA (25-350° C. at 10° C./min under nitrogen): non-crosslinked polymer: practically no mass loss up to 210° C., decomposition between 220-350° C.; crosslinked polymer: practically no mass loss up to 210° C., decomposition between 220-350° C.

### Example A13

### 3-(2-Chloro-ethyl)-2,2,5,5-tetramethyl-imidazolidin-4-one-1-N-oxyl (Cmpd Nr. 13)

Sodium hydride (55%; 1.4 g, 32 mmol) is slowly added to a suspension of 2,2,5,5-tetramethyl-imidazolidin-4-one-1-N-oxyl (4.7 g, 30 mmol) in DMF (55 ml) and the reaction mixture stirred at 25° C. for two hours. The reaction mixture is cooled with ice and 1-bromo-2-chloroethane (97%; 6.65 g, 45 mmol) are slowly fed in. The ice bath is removed and the reaction mixture stirred overnight. Ethanol is added (10 ml), the reaction mixture concentrated on a rotary evaporator and the residue dried on an oil pump. Purification of the residue by chromatography (silica gel, hexane/ethylacetate 1/1) gives 2.0 g of the title compound as orange crystals, mp. 58-59° C. Elemental analysis calcd. for C₉H₁₆ClN₂O₂ (219.69): C, 49.21%; H, 7.34%; Cl, 16.14%; N, 12.75%; found: C, 49.88%; H, 7.38%; Cl, 15.8%; N, 12.63%.

### Example A14

### 2,2,5,5-Tetramethyl-3-vinyl-imidazolidin-4-one-1-N-oxyl (Cmpd Nr. 14)

Sodium methoxide (5.4 molar in MeOH; 0.92 ml, 5.0 mmol) is slowly added at 25° C. to a stirred solution of 3-(2-chloro-ethyl)-2,2,5,5-tetramethyl-imidazolidin-4-one-1-N-oxyl (Cmpd Nr. 13, 1.0 g, 4.6 mmol) in toluene (10 ml), the progress of the reaction being monitored by GLC. Additional sodium methoxide (5.4 molar in MeOH; 0.92 ml, 5.0 mmol) is fed in after 24 hours and stirring continued for another two days. The mixture is filtered through a plug of silica gel, the solvent distilled off and the residue purified by chromatography (silica gel, hexane/ethylacetate 4/1) to afford 0.4 g of the title compound as an orange solid, mp 76-78° C. MS: for C₉H₁₅N₂O₂ (183.2) found M⁺=183.

### B) APPLICATION EXAMPLES

Abbreviations: working electrode WE; counter electrode CE; reference electrode RE; standard calomel electrode SCE; normal hydrogen electrode NHE; anodic peak potential E_{p,a}; mol/liter M.

### Cyclic Voltammetry (CV)—General Conditions:

CV is performed using a three-electrode glass cell (WE, CE, RE) and a computer-controlled potentiostat, applying a linear potential sweep (see e.g. B. Schoellhorn et al., New Journal of Chemistry, 2006, 30, 430-434; CAN144:441363). Multiple CV-scans per compound used are recorded and the mean value for the peak potential is taken. The results are presented in Table 2.

### CV—Experimental Conditions A:

### Potentiostat: 757 VA Comptrace (Metrohm)

0.1 M Bu₄NBF₄, 2.6E-3M nitroxide, MeCN
Pt disk d=5 mm (WE), Pt wire (CE), SCE (RE; +0.244V vs. NHE)
0-1.2V (TEMPO)/0-2.0V, 0.1V/s, 25° C.

### CV—Experimental Conditions B:

### Potentiostat: VersaStat II (EG&G Instruments)

0.1 M Bu₄NBF₄, 2.7E-3M nitroxide, MeCN
Pt disk d=\sqrt{5} mm (WE), Pt wire (CE), Ag/AgCl/NaCl sat'd (RE; +0.194V vs. NHE)
0-1.2V (TEMPO), 0-2.0V, 0.1V/s, 25° C.

TABLE 2

| Compound | | E_{p,a}[V]vs. | |
|---|---|---|---|
| No. | Structure | Ag/Ag⁺ | Oxidation |
| C1 comparative | 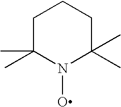 | 0.75 | reversible |
| B1 | 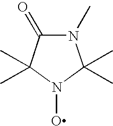 compound 1 | 1.23 | reversible |
| C2 comparative | 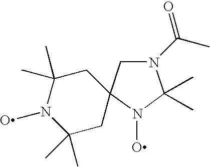 compound 2 | 1.35 | ir- reversible |
| C3 comparative | 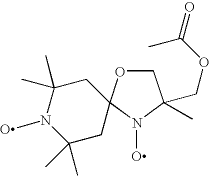 | 1.32 (vs. SCE) | ir- reversible |
| B3 | 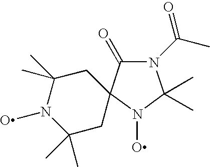 compound 6 | 1.62 | reversible |

The above CV experiments clearly indicate that the imidazolidinone compounds of the instant invention show a reversible oxidation/reduction cycle in contrast to other 5 ring heterocycles. Moreover the higher and tunable oxidation potentials of imidazolidinone nitroxides vs. TEMPO is demonstrated.

1. An electrical energy storage device with improved capacity, utilizing an electrode reaction of an active material in the reversible oxidation/reduction cycle in at least one of the positive or negative electrodes, which active material comprises a structural element of formula (I) 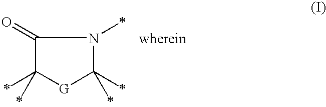
G is 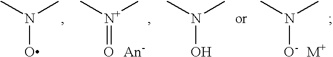
and * indicates a valence;
An⁻ is the anion of an organic or inorganic acid;
M⁺ is Li⁺;
with the proviso that the structural element of formula (I) is not attached to a 1,3,5 triazine ring.

2. An electrical energy storage device according to claim 1 wherein the structural element of formula (I) is of formulae (a1) or (a2) 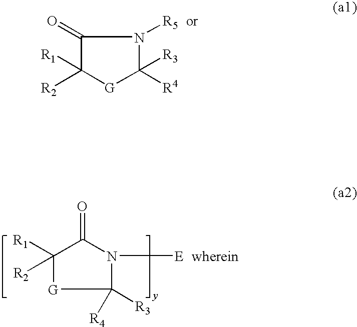
R₁, R₂, R₃ and R₄ are independently C₁-C₆alkyl, C₁-C₆alkyl substituted by —COOM⁺, —COOR₆, —CONHR₆, —CON(R₆)₂, —OR₆, F or Cl; C₁-C₆alkyl interrupted by —O— or —NR₆—; C₅-C₆cycloalkyl, C₃-C₆cycloalkylidene, C₇-C₉phenylalkyl, —COO⁻M⁺, —COOR₆, —CONHR₆, —CON(R₆)₂ or R₁ and R₂ or R₃ and R₄, or R₁ and R₂ and R₃ and R₄ are a group 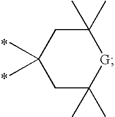
R₆ is C₁-C₆alkyl, C₁-C₆alkyl substituted by —N₃; C₂-C₆alkenyl, C₂-C₆alkinyl, glycidyl, C₅-C₆cycloalkyl, phenyl, C₇-C₉phenylalkyl or a group 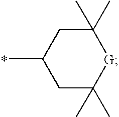
R₅ is H, C₁-C₆alkyl, C₂-C₆alkenyl, C₂-C₆alkinyl, glycidyl, C₅-C₆cycloalkyl, phenyl, C₇-C₉phenylalkyl, —O⁻M⁺, —OR₆, —OC(O)R₆, —C(O)R₆, —COOR₆, —CONHR₆, —CON(R₆)₂; or
R₅ is C₁-C₆alkyl interrupted by —O—, —NR₆— or by a group 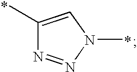
C₁-C₆alkyl, which is substituted by F, Cl, —COO⁻M⁺, —COOR₆, —CONHR₆, —CON(R₆)₂, OR₆, —OC(O)R₆, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, or P⁺(R₆)₃An⁻;
y is a number from 2 to 4;
when y is 2
E is a divalent group 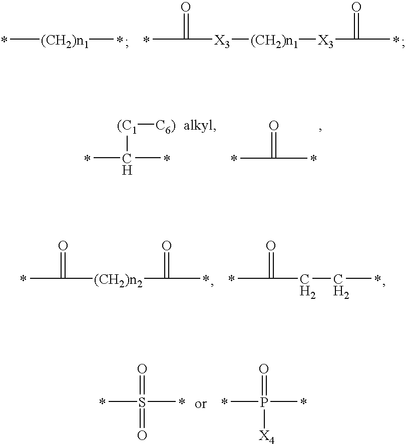
where n₁ is a number from 0 to 6 and n₂ is a number from 0 to 4; X₃ is —O—, —NH— or —NR₆; X₄ is —OR₆, —NH₂, —NHR₆, or —N(R₆)₂;
when y is 3
E is a trivalent group 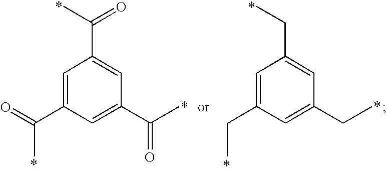
when y is 4
E is a tetravalent group of formula 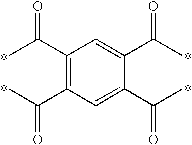
where * indicates a valence.

3. An electrical energy storage device according to claim 2 wherein
G is 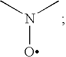
R₁, R₂, R₃ and R₄ are independently methyl, CF₃ or C₃-C₆cycloalkylidene; or
R₁ and R₂ or R₃ and R₄, or R₁ and R₂ and R₃ and R₄ are a group 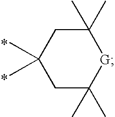
R₆ is C₁-C₆alkyl or C₂-C₆alkenyl;
R₅ is H, C₁-C₆alkyl, C₂-C₆alkenyl, C₂-C₆alkinyl, C₅-C₆cycloalkyl or —C(O)R₆; C₁-C₆alkyl, which is substituted by Cl;
y is 2;
E is a divalent group 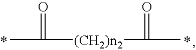
where n₂ is a number from 0 to 4;
where * indicates a valence.

4. An electrical energy storage device according to claim 1 wherein the structural element of formula (I) is the repeating unit of a polymer and is of formulae (b1), (b2), (b3), (b4) or (b5) 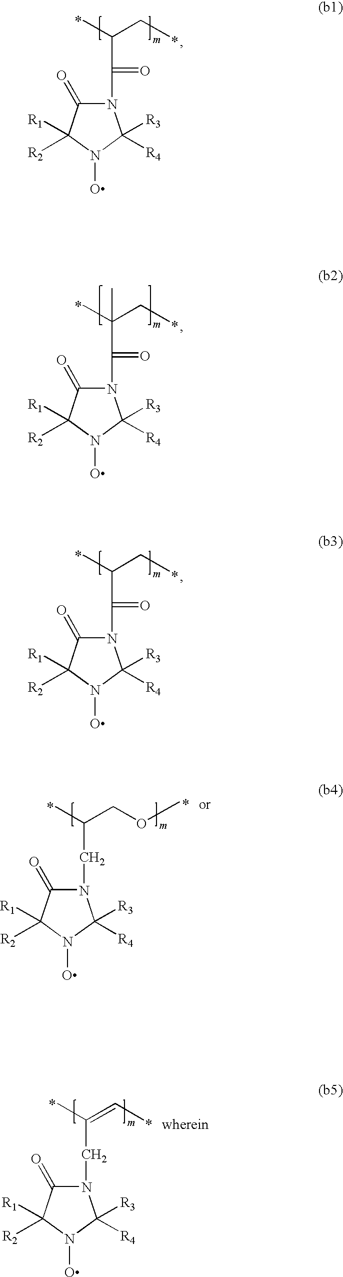
R₁, R₂, R₃ and R₄ are methyl, or C₃-C₆cycloalkylidene; or
R₁ and R₂ or R₃ and R₄, or R₁ and R₂ and R₃ and R₄ are a group 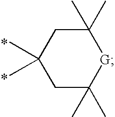
and repeating index m is a number from 2 to 50 000.

5. An electrical energy storage device according to claim 1, which is a secondary battery.

6. An electrical energy storage device according to claim 1 wherein the electrode reaction is that in the positive electrode

7. An electrical energy storage device according to claim 1 wherein the active material comprises from 10 to 100% by weight of a compound containing a structural element of formula (I).

8. An electrical energy storage device according to claim 1 wherein the active material has a spin concentration of at least 10²¹ spins/g.

9. A method for providing an electrical energy storage device with improved capacity utilizing an electrode reaction of an active material in the reversible oxidation/reduction cycle in at least one of the positive or negative electrodes, which method comprises incorporating into at least one of the positive or negative electrodes an active material comprising a structural element of formula (I) 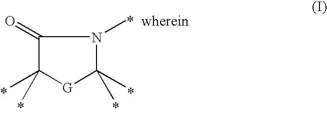
G is 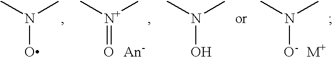
and * indicates a valence;
An⁻ is the anion of an organic or inorganic acid;
M⁺ is Li⁺;
with the proviso that the structural element of formula (I) is not attached to a 1,3,5 triazine ring.

10. (canceled)

11. A compound of formulae (a1) or (a2) 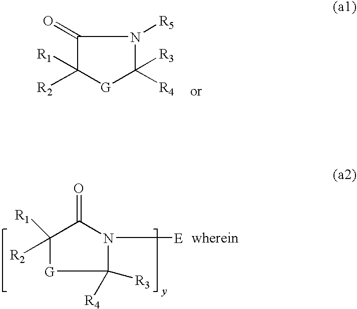
G is 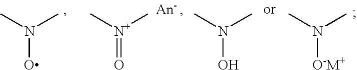
An⁻ is the anion of an organic or inorganic acid;
M⁺ is Li⁺;
R₁, R₂, R₃ and R₄ are independently —COOH, —COO(C₁-C₆alkyl) or methyl which can be substituted by F, Cl, OH, —COOH, —COO(C₁-C₆alkyl) or —O—CO(C₁-C₆alkyl);
or
R₁ and R₂ or R₃ and R₄, or R₁ and R₂ and R₃ and R₄ are a group 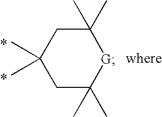
R₅ is H, C₁-C₆alkyl, C₂-C₆alkenyl, C₂-C₆alkinyl, glycidyl, C₅-C₆cycloalkyl, phenyl, C₇-C₉phenylalkyl, —O⁻M⁺, —OR₆, —OC(O)R₆, —OC(O)Cl, —C(O)Cl, —C(O)R₆, —COOR₆, —CONHR₆, —CON(R₆)₂; or
R₅ is C₁-C₆alkyl interrupted by —O—, —NR₆— or by a group 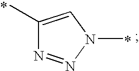
or C₁-C₆alkyl which is substituted by F, Cl, —COO⁻M⁺, —COOR₆, —CONHR₆, —CON(R₆)₂, —C(O)C₁, OR₆, —OC(O)R₆, —OC(O)OR₆, —OC(O)Cl, —OC(O)NHR₆, —OC(O)N(R₆)₂, —NHC(O)R₆, —NR₆C(O)R₆, —NCO, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, or P⁺(R₆)₃An⁻;
R₆ is —H, C₁-C₆alkyl, C₁-C₆alkyl substituted by —N₃, C₂-C₆alkenyl, C₂-C₆alkinyl, glycidyl, C₅-C₆cycloalkyl, phenyl, C₇-C₉phenylalkyl or a group 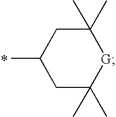
y is a number from 2 to 4;
when y is 2
E is a divalent group 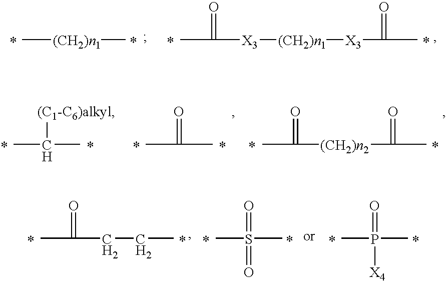
where n₁ is a number from 0 to 6 and n₂ is a number from 0 to 4; X₃ is —O—, —NH— or —NR₆—; X₄ is —OR₆, —NH₂, —NHR₆, or —N(R₆)₂;
when y is 3
E is a trivalent group 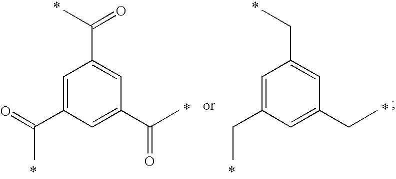
when y is 4
E is a tetravalent group of formula 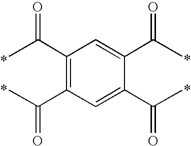
where * indicates a valence;
with the proviso that the following compounds are excluded

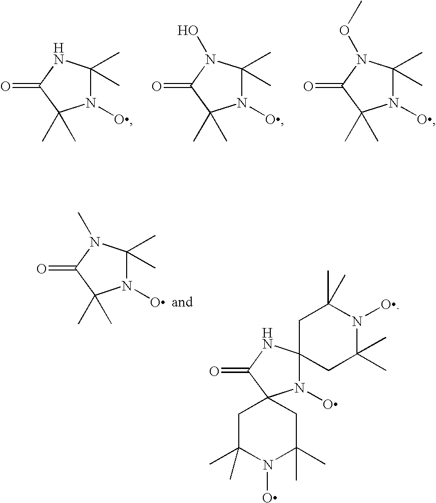

12. A compound according to claim 11 wherein
G is 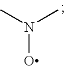
R₁, R₂, R₃ and R₄ are methyl which can be substituted by F, Cl, OH, —COOH, —COOCH₃ or —O—COCH₃; or R₁ and R₂ or R₃ and R₄, or R₁ and R₂ and R₃ and R₄ are a group 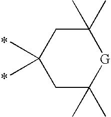
R₅ is H, C₁-C₆alkyl, C₂-C₆alkenyl, C₂-C₆alkinyl, glycidyl, C₅-C₆cycloalkyl, phenyl, C₇-C₉phenylalkyl, —OH, —OL₁, OR₆, —C(O)R₆, —OC(O)R₆, —COOR₆, —CONHR₆, —CON(R₆)₂; or
R₅ is C₁-C₆alkyl interrupted by —O— or —NR₆—; or C₁-C₆alkyl which is substituted by F, Cl, —COO⁻M⁺, —COOR₆, —CONHR₆, —CON(R₆)₂, OH, OR₆, —OC(O)R₆, —OC(O)OR₆, —OC(O)NHR₆, —OC(O)N(R₆)₂, —NHC(O)R₆, —NR₆C(O)R₆, —NCO, —N₃, NHC(O)NHR₆, —NR₆C(O)N(R₆)₂, —NHCOOR₆, —N(R₆)₂, —NR₆COOR₆, —N⁺(R₆)₃An⁻, S⁺(R₆)₂An⁻, or P⁺(R₆)₃An⁻;
R₆ is —H, C₁-C₆alkyl or C₁-C₆alkyl substituted by —N₃, C₂-C₆alkenyl, C₂-C₆alkinyl, glycidyl, C₅-C₆cycloalkyl, C₇-C₉phenylalkyl or a group 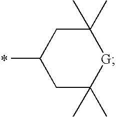
y is 2;
E is a divalent group 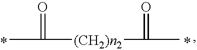
where n₂ is a number from 0 to 4.

13. A polymer with a repeating unit of formulae (b1), (b2), (b3), (b4) or (b5) 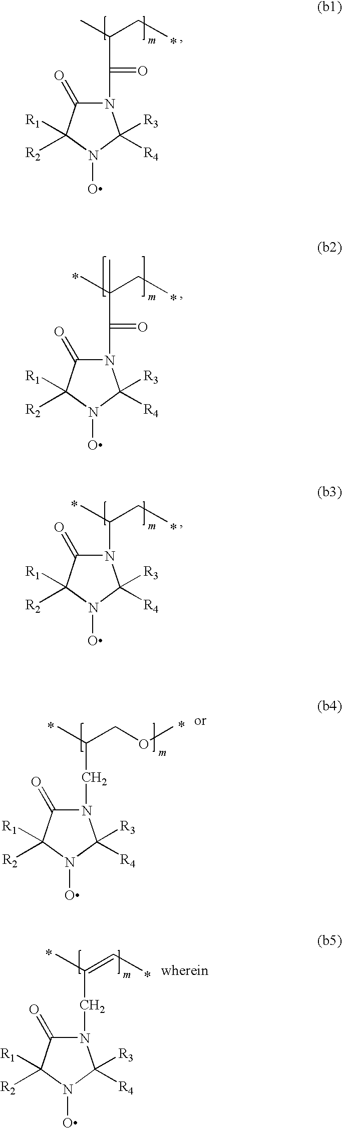
R₁, R₂, R₃ and R₄ are independently methyl, or C₃-C₆cycloalkylidene; or
R₁ and R₂ or R₃ and R₄, or R₁ and R₂ and R₃ and R₄ are a group 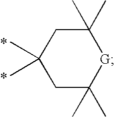
and repeating index m is a number from 2 to 50 000.
